(19) United States
(12) Patent Application Publication
(10) Pub. No.: US 2013/0317587 A1
(43) Pub. Date: Nov. 28, 2013

(54) METHODS FOR STIMULATING THE DORSAL ROOT GANGLION WITH A LEAD HAVING SEGMENTED ELECTRODES

(71) Applicant: BOSTON SCIENTIFIC NEUROMODULATION CORPORATION, Valencia, CA (US)

(72) Inventor: John Michael Barker, Ventura, CA (US)

(73) Assignee: BOSTON SCIENTIFIC NEUROMODULATION CORPORATION, Valencia, CA (US)

(21) Appl. No.: 13/899,316

(22) Filed: May 21, 2013

Related U.S. Application Data
(60) Provisional application No. 61/651,822, filed on May 25, 2012.

(51) Int. Cl. A61N 1/05

(52) U.S. Cl. CPC A61N 1/0551
USPC 607/118

(57) ABSTRACT

A method of stimulating a dorsal root ganglion includes providing an electrical stimulation lead having a distal end, a proximal end, a longitudinal length, a circumference, a plurality of electrodes disposed along the distal end of the lead, a plurality of terminals disposed along the proximal end of the lead, and a plurality of conductors. Each conductor electrically couples at least one of the electrodes to at least one of the terminals. The plurality of electrodes includes a plurality of segmented electrodes and each of the segmented electrodes extends around no more than 75% of the circumference of the lead. The method further includes implanting the electrical stimulation lead adjacent to the dorsal root ganglion and applying electrical stimulation to the dorsal root ganglion using at least one of the plurality of segmented electrodes of the electrical stimulation lead.

### CROSS-REFERENCE TO RELATED APPLICATIONS

This application claims the benefit under 35 U.S.C. §119(e) of U.S. Provisional Patent Application Ser. No. 61/651,822 filed on May 25, 2012, which is incorporated herein by reference.

### FIELD

The invention is directed to the area of electrical stimulation systems and methods of making and using the systems. The present invention is also directed to stimulating a dorsal root ganglion with an electrical stimulation lead having segmented electrodes, as well as electrical stimulation systems for performing the stimulation.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

Dorsal root ganglia are nodules of cell bodies disposed along the dorsal roots of spinal nerves. Dorsal root ganglia are disposed external to the epidural space. Dorsal root ganglia, however, are disposed in proximity to the spinal cord and the vertebral column.

### BRIEF SUMMARY

One embodiment is a method of stimulating a dorsal root ganglion. The method includes providing an electrical stimulation lead having a distal end, a proximal end, a longitudinal length, a circumference, a plurality of electrodes disposed along the distal end of the lead, a plurality of terminals disposed along the proximal end of the lead, and a plurality of conductors. Each conductor electrically couples at least one of the electrodes to at least one of the terminals. The plurality of electrodes includes a plurality of segmented electrodes and each of the segmented electrodes extends around no more than 75% of the circumference of the lead. The method further includes implanting the electrical stimulation lead adjacent to the dorsal root ganglion and applying electrical stimulation to the dorsal root ganglion using at least one of the plurality of segmented electrodes of the electrical stimulation lead.

Another embodiment is an electrical stimulation lead that includes a lead body having a distal end, a proximal end, a longitudinal length, and a circumference; a plurality of electrodes disposed along the distal end of the lead body; a plurality of terminals disposed along the proximal end of the lead body; and a plurality of conductors, each conductor electrically coupling at least one of the electrodes to at least one of the terminals. The plurality of electrodes includes a plurality of segmented electrodes and each of the segmented electrodes extends around no more than 75% of the circumference of the lead body. The electrical stimulation lead is configured and arranged for implantation near, and stimulation of, a dorsal root ganglion.

Yet another embodiment is an electrical stimulation system including the electrical stimulation lead described above; and a control module coupleable to the electrical stimulation lead and configured and arranged for providing stimulation current to patient tissue via the electrical stimulation lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2A is a schematic view of one embodiment of a proximal portion of a lead and a control module of an electrical stimulation system, according to the invention;

FIG. 2B is a schematic view of one embodiment of a proximal portion of a lead and a lead extension of an electrical stimulation system, according to the invention;

FIG. 3A is a schematic transverse cross-sectional view of spinal nerves extending from a spinal cord, the spinal nerves including dorsal root ganglia;

FIG. 3B is a schematic perspective view of a portion of the spinal cord of FIG. 3A disposed in a portion of a vertebral column with the dorsal root ganglia of FIG. 3A extending outward from the vertebral column;

FIG. 3C is a schematic top view of a portion of the spinal cord of FIG. 3A disposed in a vertebral foramen defined in a vertebra of the vertebral column of FIG. 3B, the vertebra also defining intervertebral foramina extending between an outer surface of the vertebra and the vertebral foramen, the intervertebral foramina providing an opening through which the dorsal root ganglia of FIG. 3B can extend outward from the spinal cord of FIG. 3B;

FIG. 3D is a schematic side view of two vertebrae of the vertebral column of FIG. 3B, the vertebrae defining an intervertebral foramen through which the dorsal root ganglia of FIG. 3B can extend outward from the spinal cord of FIG. 3B;

FIG. 4 is a schematic perspective view of the distal end of one embodiment of a lead with segmented electrodes, according to the invention;

FIG. 5A is a schematic perspective view of the distal end of a second embodiment of a lead with segmented electrodes, according to the invention;

FIG. 5B is a schematic perspective view of the distal end of a third embodiment of a lead with segmented electrodes, according to the invention;

FIG. 5C is a schematic perspective view of the distal end of a fourth embodiment of a lead with segmented electrodes, according to the invention;

FIG. 5D is a schematic side view of the distal end of a fifth embodiment of a lead with segmented electrodes, according to the invention;

FIG. 5E is a schematic side view of the distal end of a sixth embodiment of a lead with segmented electrodes, according to the invention;

FIG. 5F is a schematic side view of the distal end of a seventh embodiment of a lead with segmented electrodes, according to the invention;

FIG. 6A is a schematic perspective view of one embodiment of a lead implanted near a dorsal root ganglion, according to the invention;

FIG. 6B is a schematic perspective view of a second embodiment of a lead implanted near a dorsal root ganglion, according to the invention;

FIG. 6C is a schematic perspective view of one embodiment of a lead having distal end with a hook shape disposed around a dorsal root ganglion, according to the invention;

FIG. 6D is a schematic perspective view of one embodiment of a lead having distal end with a coil shape disposed around a dorsal root ganglion, according to the invention; and

FIG. 7 is a schematic overview of one embodiment of components of an electrical stimulation system, according to the invention.

### DETAILED DESCRIPTION

The invention is directed to the area of electrical stimulation systems and methods of making and using the systems. The present invention is also directed to stimulating a dorsal root ganglion with an electrical stimulation lead having segmented electrodes, as well as electrical stimulation systems for performing the stimulation.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; and 8,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; and 2012/0316615; and U.S. patent application Ser. Nos. 12/177,823; 13/667,953; and 13/750,725, all of which are incorporated by reference.

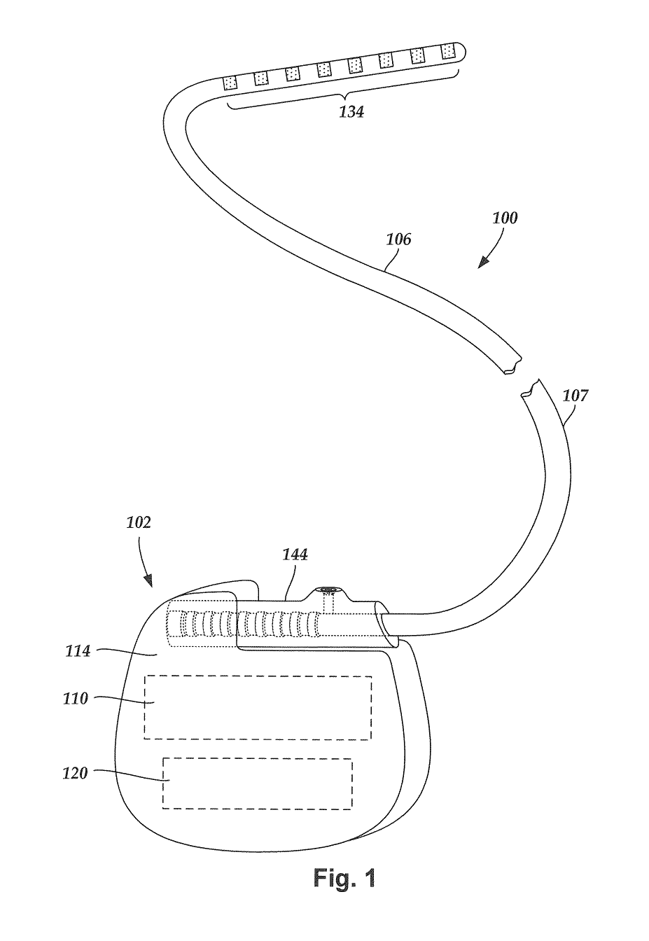

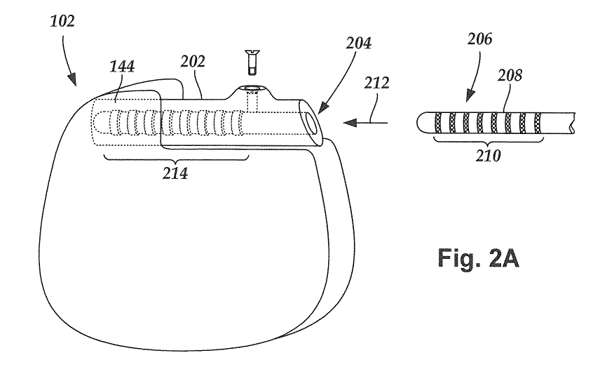

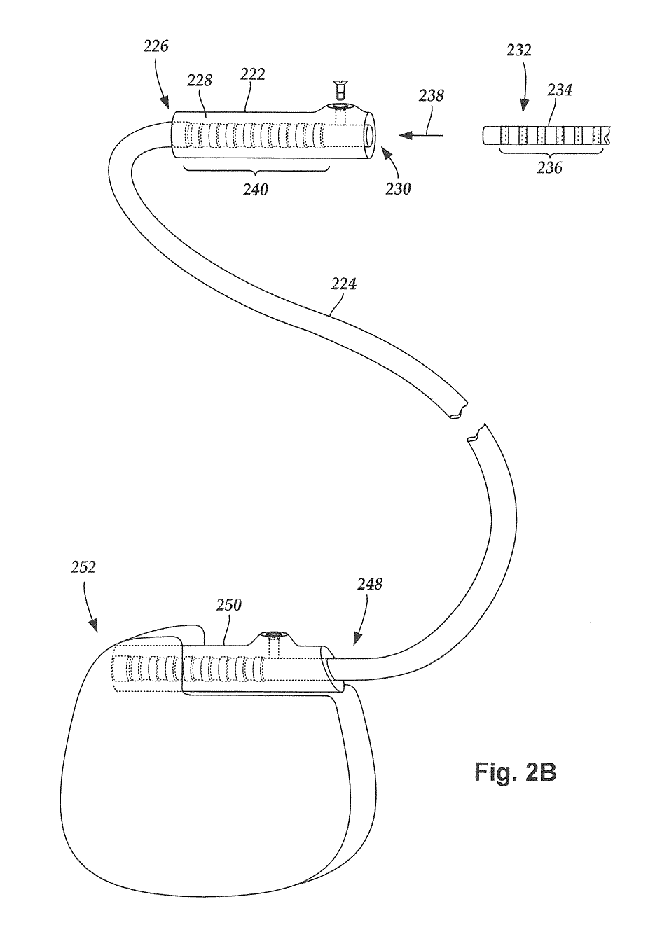

FIG. 1 illustrates schematically one embodiment of an electrical stimulation system 100. The electrical stimulation system includes a control module (e.g., a stimulator or pulse generator) 102 and at least one lead 106 coupled to the control module 102. Each lead 106 typically includes a lead body 107 and an array of electrodes 134. The control module 102 typically includes an electronic subassembly 110 and an optional power source 120 disposed in a sealed housing 114. The control module 102 typically includes a connector 144 (FIG. 2A, see also 222 and 250 of FIG. 2B) into which the proximal end of the one or more leads 106 can be plugged to make an electrical connection via conductive contacts on the control module 102 and terminals (e.g., 210 in FIG. 2A and 236 of FIG. 2B) on each of the one or more leads 106. In at least some embodiments, a lead is isodiametric along a longitudinal length of the lead 106. In addition, one or more lead extensions 224 (see FIG. 2B) can be disposed between the one or more leads 106 and the control module 102 to extend the distance between the one or more leads 106 and the control module 102 of the embodiment shown in FIG. 1.

The electrical stimulation system or components of the electrical stimulation system, including one or more of the leads 106 and the control module 102, are typically implanted into the body of a patient. The electrical stimulation system can be used for a variety of applications including, but not limited to, electrical stimulation of the dorsal root ganglia.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. The number of electrodes 134 in the array of electrodes 134 may vary. For example, there can be two, four, six, eight, ten, twelve, fourteen, sixteen, or more electrodes 134. As will be recognized, other numbers of electrodes 134 may also be used.

The electrodes of one or more leads 106 are typically disposed in, or separated by, a non-conductive, biocompatible material such as, for example, silicone, polyurethane, polyetheretherketone (“PEEK”), epoxy, and the like or combinations thereof. The leads 106 may be formed in the desired shape by any process including, for example, molding (including injection molding), casting, and the like. The non-conductive material typically extends from the distal end of the one or more leads 106 to the proximal end of each of the one or more leads 106 and forms a lead body 107.

Terminals (e.g., 210 in FIG. 2A and 236 of FIG. 2B) are typically disposed at the proximal end of the one or more leads 106 of the electrical stimulation system 100 for connection to corresponding conductive contacts (e.g., 214 in FIG. 2A and 240 of FIG. 2B) in connectors (e.g., 144 in FIGS. 1-2A and 222 and 250 of FIG. 2B) disposed on, for example, the control module 102 (or to conductive contacts on a lead extension, an operating room cable, or an adaptor). Conductor wires (not shown) extend from the terminals (e.g., 210 in FIG. 2A and 236 of FIG. 2B) to the electrodes 134. Typically, one or more electrodes 134 are electrically coupled to a terminal (e.g., 210 in FIG. 2A and 236 of FIG. 2B). In at least some embodiments, each terminal (e.g., 210 in FIG. 2A and 236 of FIG. 2B) is only connected to one electrode 134.

The conductor wires may be embedded in the non-conductive material of the lead 106 or can be disposed in one or more lumens (not shown) extending along the lead 106. In some embodiments, there is an individual lumen for each conductor wire. In other embodiments, two or more conductor wires may extend through a lumen. There may also be one or more lumens (not shown) that open at, or near, the proximal end of the lead 106, for example, for inserting a stylet wire to facilitate placement of the lead 106 within a body of a patient. Additionally, there may also be one or more lumens (not shown) that open at, or near, the distal end of the lead 106, for example, for infusion of drugs or medication into the site of implantation of the one or more leads 106. In at least one embodiment, the one or more lumens may be flushed continually, or on a regular basis, with saline, epidural fluid, or the like. In at least some embodiments, the one or more lumens can be permanently or removably sealable at the distal end.

In at least some embodiments, leads are coupled to connectors disposed on control modules. In FIG. 2A, a lead 208 is shown configured and arranged for insertion to the control module 102. The connector 144 includes a connector housing 202. The connector housing 202 defines at least one port 204 into which a proximal end 206 of a lead 208 with terminals 210 can be inserted, as shown by directional arrow 212. The connector housing 202 also includes a plurality of conductive contacts 214 for each port 204. When the lead 208 is inserted into the port 204, the conductive contacts 214 can be aligned with the terminals 210 on the lead 208 to electrically couple the control module 102 to the electrodes (134 of FIG. 1) disposed at a distal end of the lead 208. Examples of connectors in control modules are found in, for example, U.S. Pat. Nos. 7,244,150 and 8,224,450, which are incorporated by reference.

In FIG. 2B, a connector 222 is disposed on a lead extension 224. The connector 222 is shown disposed at a distal end 226 of the lead extension 224. The connector 222 includes a connector housing 228. The connector housing 228 defines at least one port 230 into which a proximal end 232 of a lead 234 with terminals 236 can be inserted, as shown by directional arrow 238. The connector housing 228 also includes a plurality of conductive contacts 240. When the lead 234 is inserted into the port 230, the conductive contacts 240 disposed in the connector housing 228 can be aligned with the terminals 236 on the lead 234 to electrically couple the lead extension 224 to the electrodes (134 of FIG. 1) disposed at a distal end (not shown) of the lead 234.

In at least some embodiments, the proximal end of a lead extension is similarly configured and arranged as a proximal end of a lead. The lead extension 224 may include a plurality of conductive wires (not shown) that electrically couple the conductive contacts 240 to a proximal end 248 of the lead extension 224 that is opposite to the distal end 226. In at least some embodiments, the conductive wires disposed in the lead extension 224 can be electrically coupled to a plurality of terminals (not shown) disposed on the proximal end 248 of the lead extension 224. In at least some embodiments, the proximal end 248 of the lead extension 224 is configured and arranged for insertion into a connector disposed in another lead extension. In other embodiments, the proximal end 248 of the lead extension 224 is configured and arranged for insertion into a connector disposed in a control module. As an example, in FIG. 2B the proximal end 248 of the lead extension 224 is inserted into a connector 250 disposed in a control module 252.

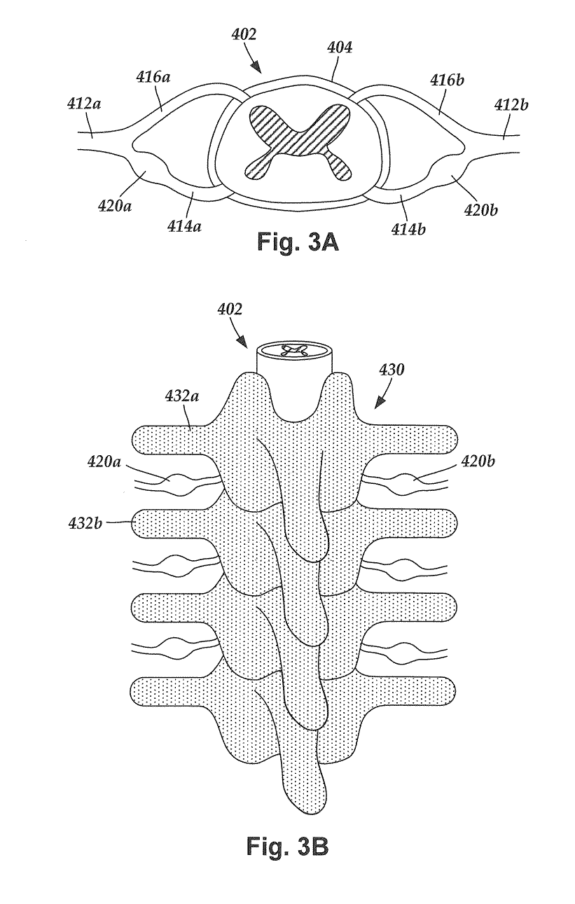

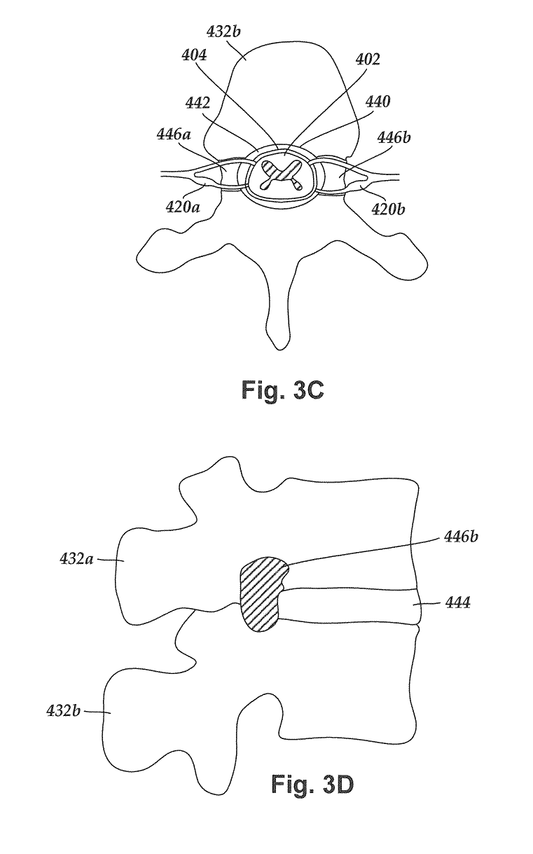

Turning to FIG. 3A, one potential target stimulation location is the dorsal root ganglia. FIG. 3A schematically illustrates a transverse cross-sectional view of a spinal cord 402 surrounded by dura 404. The spinal cord 402 includes a plurality of levels from which spinal nerves 412a and 412b extend. In at least some spinal cord levels, the spinal nerves 412a and 412b extend bilaterally from the spinal cord 402. In FIG. 3A, the spinal nerves 412a and 412b attach to the spinal cord 402 via corresponding dorsal roots 414a and 414b and corresponding ventral (or anterior) roots 416a and 416b. Typically, the dorsal roots 414a and 414b relay sensory information into the spinal cord 402 and the ventral roots 416a and 416b relay motor information outward from the spinal cord 402. Dorsal root ganglia (“DRG”) 420a and 420b are nodules of cell bodies that are disposed along the dorsal roots 416a and 416b in proximity to the spinal cord 402.

FIG. 3B schematically illustrates a perspective view of a portion of the spinal cord 402 disposed along a portion of a vertebral column 430. The vertebral column 430 includes a plurality of stacked vertebrae, such as vertebrae 432a and 432b, and a plurality of DRGs 420a and 420b extending outwardly bilaterally from the spinal cord 402.

FIG. 3C schematically illustrates a top view of a portion of the spinal cord 402 and dura 404 disposed in a vertebral foramen 440 defined in the vertebra 432b. The vertebrae 432 are stacked together and the vertebral foramina 440 of the vertebrae collectively form a spinal canal through which the spinal cord 402 extends. The space within the spinal canal between the dura 404 and the walls of the vertebral foramen 440 defines the epidural space 442. Intervertebral foramina 446a and 446b defined bilaterally along sides of the vertebra 432b form openings through the vertebra 432b between the epidural space 442 and the environment external to the vertebra 432b.

FIG. 3D schematically illustrates a side view of two vertebrae 432a and 432b coupled to one another by a disc 444. In FIG. 3D, the intervertebral foramen 446b is shown defined between the vertebrae 432a and 432b. The intervertebral foramen 446b provides an opening for one or more of the dorsal root 414b, ventral root 416b, and DRG 420b to extend outwardly from the spinal cord 402.

Stimulation electrodes 134 are disposed along the lead 106 to stimulate the target tissue, such as the dorsal root ganglion. Although the electrodes 134 can have any suitable shape, including, but not limited to, ring electrodes, tip electrodes, and segmented electrodes, at least some of the electrodes 134 are segmented electrodes. Electrodes that are ring-shaped typically project current equally in every direction from the position of the electrode along a length of the lead 106. Ring electrodes, by themselves, typically do not enable stimulus current to be directed to only one side of the lead. Segmented electrodes, however, can be used to direct stimulus current to one side, or even a portion of one side, of the lead. The use of segmented electrodes may be beneficial to more directly target the DRG and, at least in some cases, to reduce the inadvertent stimulation of other tissue, including other nerve or spinal cord tissue, in the neighborhood of the DRG. Inadvertent stimulation of the other tissue (for example, the anterior root, the spinal cord, the dorsal root ganglion at a different spinal level, and the like) may result in side-effects which may be deleterious.

It will be understood that, in at least some embodiments, a segmented electrode may be used in conjunction with a ring electrode as a cathode-anode pair to provide stimulation directed to target tissue adjacent the segmented electrode. Examples of leads with segmented electrodes include U.S. Pat. Nos. 8,295,944; and 8,391,985; and U.S. Patent Applications Publication Nos. 2010/0268298; 2011/0005069; 2011/0078900; 2011/0130817; 2011/0130818; 2011/0238129; 2011/0313500; 2012/0016378; 2012/0046710; 2012/0165911; 2012/0197375; 2012/0203316; 2012/0203320; and 2012/0203321, all of which are incorporated herein by reference.

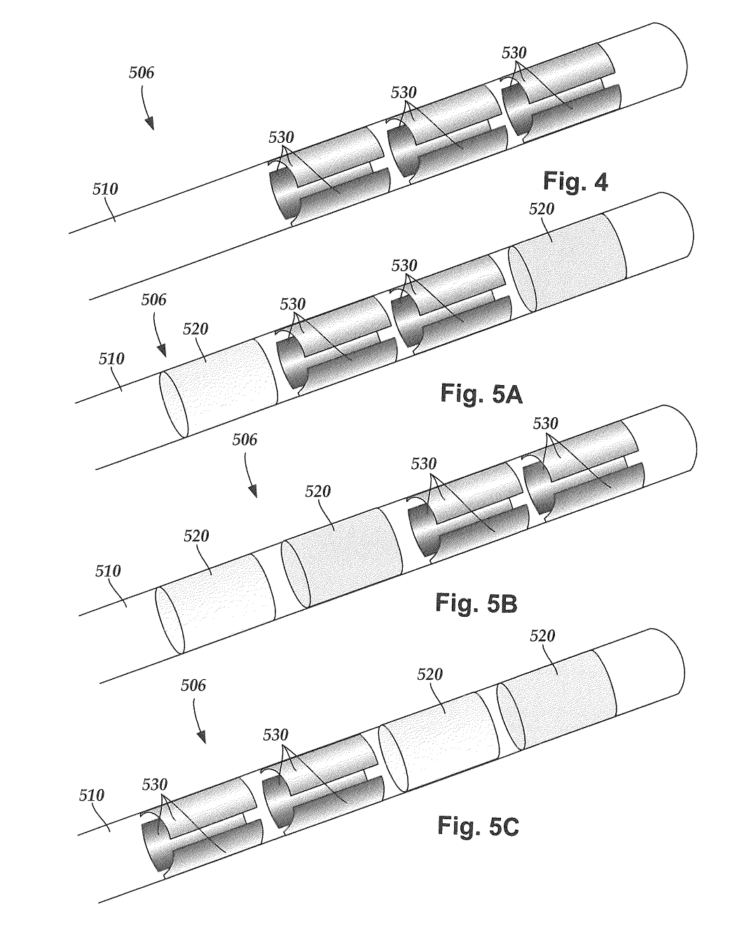

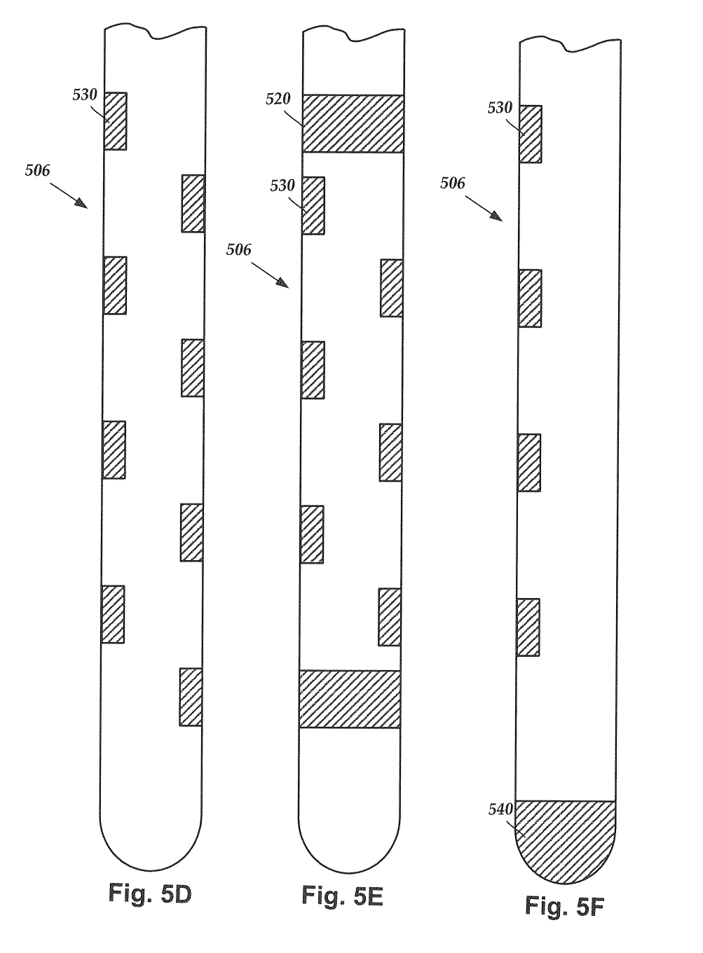

FIG. 4 illustrates one embodiment of a distal portion of a lead 506 for electrical stimulation of patient tissue, such as the dorsal root ganglia. The lead 506 includes a lead body 510 and a plurality of segmented electrodes 530 disposed along the distal portion of the lead. Other embodiments may also contain one or more ring electrodes (see, for example, FIGS. 5A-5C and 5E) or a tip electrode or any combination thereof. The lead body 510 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 506 may be in contact with body tissue for extended periods of time. In at least some embodiments, the lead 506 has a cross-sectional diameter of no more than 1.5 mm and may be in the range of 1 to 3 mm. In at least some embodiments, the lead 506 has a length of at least 10 cm and the length of the lead 506 may be in the range of 25 to 70 cm.

Each of the electrodes can either be used or unused (OFF). When the electrode is used, the electrode can be used as an anode or cathode and carry anodic or cathodic current. In some instances, an electrode might be an anode for a period of time and a cathode for a period of time.

Any number of segmented electrodes 530 may be disposed on the lead body 510 including, for example, one, two three, four, five, six, seven, eight, nine, ten, eleven, twelve, thirteen, fourteen, fifteen, sixteen or more segmented electrodes 530. It will be understood that any number of segmented electrodes 530 may be disposed along the length of the lead body 510. In at least some embodiments, each segmented electrode extends no more than 75%, 50%, 33%, 30%, 25%, 20%, or 15% around the circumference of the lead.

In at least some embodiments, the segmented electrodes 530 are arranged in sets of segmented electrodes with each set being positioned around the circumference of the lead body 510 at a particular longitudinal position along the lead, as illustrated, for example, in FIGS. 4 and 5A-5C. An advantage of using these sets of segmented electrodes is that the practitioner can select which electrodes from a set or sets to use for stimulation. Moreover, the practitioner may have less concern regarding whether the segmented electrodes are positioned properly for stimulation of the DRG or other target tissue because at least one segmented electrode of each is set is likely to be properly positioned adjacent the tissue to be stimulated. Markers or other indicia may be provided sot that the practitioner can determine the orientation of the segmented electrodes when implanted. Examples of suitable markers and indicia can be found in, for example, U.S. Patent Applications Publication Nos. 2012/0016378 and 2012/0203321; and U.S. patent application Ser. Nos. 13/750,725 and 13/787,171, all of which are incorporated herein by reference.

The lead 506 may have any number of segmented electrodes 530 in a given set of segmented electrodes. The lead 506 may have one, two, three, four, five, six, seven, eight, or more segmented electrodes 530 in a given set. In at least some embodiments, each set of segmented electrodes 530 of the lead 506 contains the same number of segmented electrodes 530. The segmented electrodes 530 disposed on the lead 506 may include a different number of electrodes than at least one other set of segmented electrodes 530 disposed on the lead 506.

Each set of segmented electrodes 530 may be disposed around the circumference of the lead body 510 to form a substantially cylindrical shape around the lead body 510. The spacing between individual electrodes of a given set of the segmented electrodes may be the same, or different from, the spacing between individual electrodes of another set of segmented electrodes on the lead 506. In at least some embodiments, equal spaces, gaps or cutouts are disposed between each segmented electrode 530 around the circumference of the lead body 510. In other embodiments, the spaces, gaps or cutouts between the segmented electrodes 530 may differ in size or shape. In other embodiments, the spaces, gaps, or cutouts between segmented electrodes 530 may be uniform for a particular set of the segmented electrodes 530, or for all sets of the segmented electrodes 530. The sets of segmented electrodes 530 may be positioned in irregular or regular intervals along a length the lead body 510.

In at least some embodiments, the segmented electrodes 530 (or a subset of the segmented electrodes) are not arranged in sets of segmented electrodes. FIGS. 5D-5F illustrate examples of such arrangements. It will be understood that the segmented electrodes can be arranged in any desired configuration around the distal end of the lead. FIGS. 5D and 5E illustrate leads 506 with segmented electrodes 530 arranged in one or more helices disposed around the circumference of the lead body 510. FIG. 5F illustrates a lead 506 with segmented electrodes 530 arranged on only one side of the lead.

The segmented electrodes 530 may vary in size and shape. In some embodiments, the segmented electrodes 530 are all of the same size, shape, diameter, width or area or any combination thereof. In some embodiments, the segmented electrodes 530 of each circumferential set (or even all segmented electrodes disposed on the lead 506) may be identical in size and shape.

The set of segmented electrodes 530 can be aligned in any arrangement with respect to each other. For example, the segmented electrodes 530 may be aligned with the segmented electrodes 530 of one or more other sets (for example, the adjacent set(s)), as illustrated in FIGS. 4 and 5A-5C. Alternatively or additionally, the segmented electrodes 530 may be staggered or angularly offset around the circumference of the lead with respect to the segmented electrodes of one or more other sets.

In at least some embodiments, electrodes in the form of ring electrodes 520 may be disposed on any part of the lead body 510, usually near a distal end of the lead 506, as illustrated, for example, in FIGS. 5A-5C and 5E. In FIGS. 5A-5C and 5E, the lead 506 includes two ring electrodes 520. Any number of ring electrodes 520 may be disposed along the length of the lead body 510 including, for example, one, two three, four, five, six, seven, eight, nine, ten, eleven, twelve, thirteen, fourteen, fifteen, sixteen or more ring electrodes 520. It will be understood that any number of ring electrodes may be disposed along the length of the lead body 510. In some embodiments, the ring electrodes 520 are substantially cylindrical and wrap around the entire circumference of the lead body 510. In some embodiments, the outer diameters of the ring electrodes 520 are substantially equal to the outer diameter of the lead body 510. The length of the ring electrodes 520 may vary according to the desired treatment and the location of the target neurons. In some embodiments the length of the ring electrodes 520 are less than or equal to the diameters of the ring electrodes 520. In other embodiments, the lengths of the ring electrodes 520 are greater than the diameters of the ring electrodes 520. In some embodiments, the lead may include a tip electrode 540 (see, FIG. 5F) which can be similar to a ring electrode except that it covers the distal tip of the lead.

Conductor wires that attach to the ring electrodes 520 or segmented electrodes 530 extend along the lead body 510. These conductor wires may extend through the material of the lead 506 or along one or more lumens defined by the lead 506, or both. The conductor wires are presented at a connector (via terminals) for coupling of the electrodes 520, 530 to a control unit (not shown).

When the lead 506 includes both ring electrodes 520 and segmented electrodes 530, the ring electrodes 520 and the segmented electrodes 530 may be arranged in any suitable configuration. For example, when the lead 506 includes two sets of ring electrodes 520 and two sets of segmented electrodes 530, the ring electrodes 520 can flank the two sets of segmented electrodes 530 (see e.g., FIGS. 5A and 5E). Alternately, the two sets of ring electrodes 520 can be disposed proximal to the two sets of segmented electrodes 530 (see, for example, FIG. 5B), or the two sets of ring electrodes 520 can be disposed distal to the two sets of segmented electrodes 530 (see, for example, FIG. 5C). It will be understood that other configurations are possible as well (e.g., alternating ring and set of segmented electrodes, or the like). Alternatively or additionally, a tip electrode 540 (see, for example, FIG. 5F) can be used as the distal-most electrode.

By varying the location of the segmented electrodes 530, different coverage of the target tissue may be selected. For example, the electrode arrangement of FIG. 5B may be useful if the physician anticipates that the target will be closer to a distal tip of the lead body 510, while the electrode arrangement of FIG. 5C may be useful if the physician anticipates that the neural target will be closer to a proximal end of the lead body 510.

Any combination of ring electrodes 520 and segmented electrodes 530 may be disposed on the lead 506. For example, the lead may include a first ring electrode, two sets of segmented electrodes, each set formed of three segmented electrodes 530, and a final ring electrode at the end of the lead, as illustrated in FIG. 5A. This configuration may simply be referred to as a 1-3-3-1 configuration. It may be useful to refer to the electrodes with this shorthand notation. Thus, the embodiment of FIG. 5B may be referred to as a 1-1-3-3 configuration, while the embodiment of FIG. 5C may be referred to as a 3-3-1-1 configuration. Other eight-electrode configurations include, for example, a 2-2-2-2 configuration, where four sets of segmented electrodes are disposed on the lead, and a 4-4 configuration, where two sets of segmented electrodes, each having four segmented electrodes 530 are disposed on the lead. In some embodiments, the lead includes 16 electrodes. Possible configurations for a 16-electrode lead include, but are not limited to 4-4-4-4; 8-8; 3-3-3-3-3-1 (and all rearrangements of this configuration); and 2-2-2-2-2-2-2-2. Using this notation, the electrode arrangement of FIG. 4 would be 3-3-3.

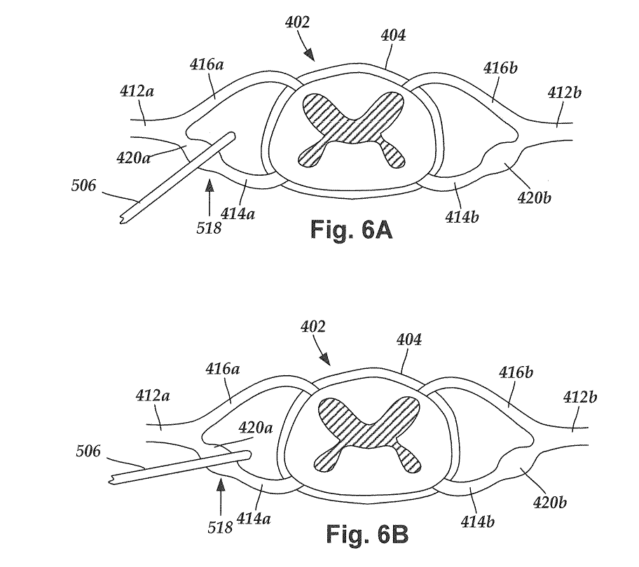

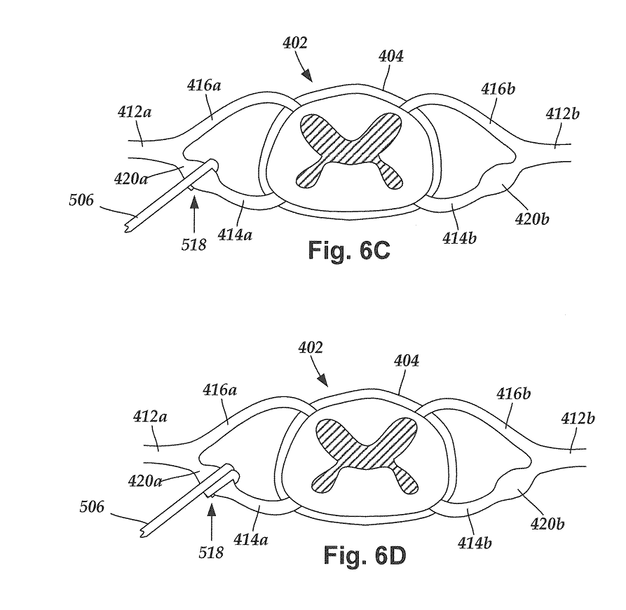

FIGS. 6A-6E illustrate a variety of different implantation arrangements for a distal end 518 of the electrical stimulation lead 506 with respect to the dorsal root ganglion 420a. For purposes of clarity, the lead electrodes are not illustrated in FIGS. 6A-6E, but it will be understood that each of the leads contain segmented electrodes, at least some of which are positioned adjacent the DRG 420a. FIG. 6A illustrates one embodiment of a lead 506 with a distal end 518 having a linear or curved shape that lies next to the DRG 420a. In these embodiments, the lead forms an angle of at least 45°, 50°, 60°, 70°, 80°, or 85° with the dorsal root 414a.

FIG. 6B illustrates one embodiment of a lead 506 with a distal end 518 having a linear or curved shape that lies next to the DRG 420a. In these embodiments, the lead forms an angle of no more than 45°, 30°, 20°, 15°, 10°, or 5° with the dorsal root 414a.

FIG. 6C illustrates one embodiment of a lead 506 with a distal end 508 of the lead having a hook-shaped distal end 518 to fit around the DRG 420a. In at least some embodiments, the hook-shaped distal end extends around at least 40%, 50%, 60%, 70%, 75%, 80%, 90%, 95%, or 100% of the circumference of the DRG 420a.

FIG. 6D illustrates one embodiment of a lead 506 with a distal end 508 of the lead having a coil-shaped distal end 518 to fit around a portion of the DRG 420a. The coil-shaped distal end may include any number of full turns (360° turn) around the DRG 420a including, for example, at least one, two, or three full turns. The coil-shaped distal end may also include a partial turn (less than 360° turn). The turns of the coil-shaped distal end may be situated immediately adjacent to each other in a touching arrangement, as illustrated in FIG. 6E, or the turns may be separated from each other or any combination thereof.

In at least some embodiments of the arrangements exemplified by FIGS. 6C and 6D, the portion of the lead extending from the hook-shaped or coil-shaped distal end is arranged to form an angle of at least 45°, 50°, 60°, 70°, 80°, or 85° with the dorsal root 414a. In at least some embodiments, the hook-shaped or coil-shaped distal end of the lead body is isodiametric. In at least some embodiments, the hook-shaped or coil-shaped distal end of the lead body is also isodiametric with the remainder of the lead. Further description of leads with hook-shaped or coiled-shaped distal end can be found in U.S. Provisional Patent Application Ser. No. 61/651,830, incorporated herein by reference.

The leads described herein can be implanted using any suitable implantation method. Novel methods and arrangements for implanting leads with segmented electrodes, as described herein, are presented in U.S. Provisional Patent Application Serial No. 61/651,815; U.S. Provisional Patent Application Ser. No. 61/651,917; and U.S. Provisional Patent Application Ser. No. 61/651,840, all of which are incorporated herein by reference.

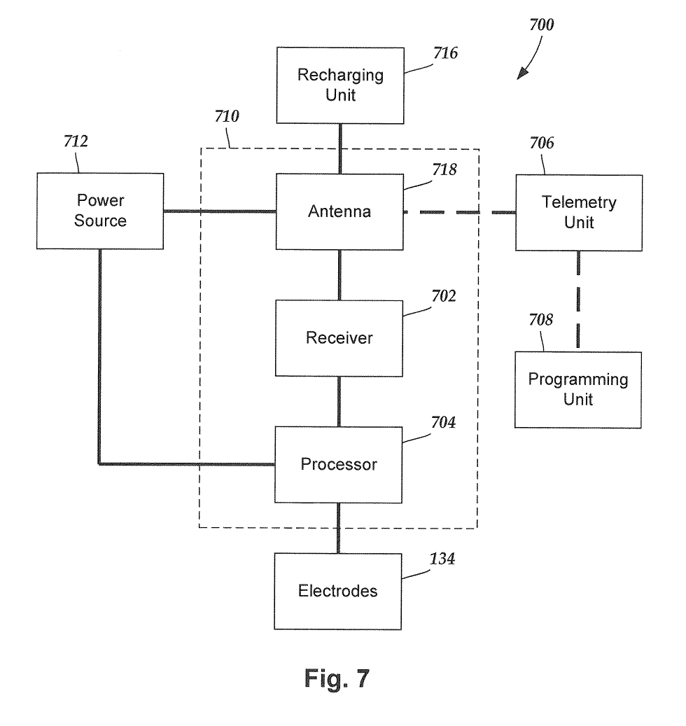

FIG. 7 is a schematic overview of one embodiment of components of an electrical stimulation system 700 including an electronic subassembly 710 disposed within a control module. It will be understood that the electrical stimulation system can include more, fewer, or different components and can have a variety of different configurations including those configurations disclosed in the stimulator references cited herein.

Some of the components (for example, power source 712, antenna 718, receiver 702, and processor 704) of the electrical stimulation system can be positioned on one or more circuit boards or similar carriers within a sealed housing of an implantable pulse generator, if desired. Any power source 712 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 718 or a secondary antenna. The external power source can be in a device that is mounted on the skin of the user or in a unit that is provided near the user on a permanent or periodic basis.

If the power source 712 is a rechargeable battery, the battery may be recharged using the optional antenna 718, if desired. Power can be provided to the battery for recharging by inductively coupling the battery through the antenna to a recharging unit 716 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 704 is generally included to control the timing and electrical characteristics of the electrical stimulation system. For example, the processor 704 can, if desired, control one or more of the timing, frequency, strength, duration, and waveform of the pulses. In addition, the processor 704 can select which electrodes can be used to provide stimulation, if desired. In some embodiments, the processor 704 may select which electrode(s) are cathodes and which electrode(s) are anodes. In some embodiments, the processor 704 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 708 that, for example, allows modification of pulse characteristics. In the illustrated embodiment, the processor 704 is coupled to a receiver 702 which, in turn, is coupled to the optional antenna 718. This allows the processor 704 to receive instructions from an external source to, for example, direct the pulse characteristics and the selection of electrodes, if desired.

In one embodiment, the antenna 718 is capable of receiving signals (e.g., RF signals) from an external telemetry unit 706 which is programmed by a programming unit 708. The programming unit 708 can be external to, or part of, the telemetry unit 706. The telemetry unit 706 can be a device that is worn on the skin of the user or can be carried by the user and can have a form similar to a pager, cellular phone, or remote control, if desired. As another alternative, the telemetry unit 706 may not be worn or carried by the user but may only be available at a home station or at a clinician's office. The programming unit 708 can be any unit that can provide information to the telemetry unit 706 for transmission to the electrical stimulation system 700. The programming unit 708 can be part of the telemetry unit 706 or can provide signals or information to the telemetry unit 706 via a wireless or wired connection. One example of a suitable programming unit is a computer operated by the user or clinician to send signals to the telemetry unit 706.

The signals sent to the processor 704 via the antenna 718 and receiver 702 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 700 to cease operation, to start operation, to start charging the battery, or to stop charging the battery. In other embodiments, the stimulation system does not include an antenna 718 or receiver 702 and the processor 704 operates as programmed.

Optionally, the electrical stimulation system 700 may include a transmitter (not shown) coupled to the processor 704 and the antenna 718 for transmitting signals back to the telemetry unit 706 or another unit capable of receiving the signals. For example, the electrical stimulation system 700 may transmit signals indicating whether the electrical stimulation system 700 is operating properly or not or indicating when the battery needs to be charged or the level of charge remaining in the battery. The processor 704 may also be capable of transmitting information about the pulse characteristics so that a user or clinician can determine or verify the characteristics.

The above specification, examples, and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A method of stimulating a dorsal root ganglion, the method comprising:
providing an electrical stimulation lead having a distal end, a proximal end, a longitudinal length, a circumference, a plurality of electrodes disposed along the distal end of the lead, a plurality of terminals disposed along the proximal end of the lead, and a plurality of conductors, each conductor electrically coupling at least one of the electrodes to at least one of the terminals, wherein the plurality of electrodes comprises a plurality of segmented electrodes, each of the segmented electrodes extending around no more than 75% of the circumference of the lead;
implanting the electrical stimulation lead adjacent to the dorsal root ganglion; and
applying electrical stimulation to the dorsal root ganglion using at least one of the plurality of segmented electrodes of the electrical stimulation lead.

2. The method of claim 1, wherein each of the plurality of electrodes is a segmented electrode.

3. The method of claim 1, wherein at least some of the segmented electrodes are formed into a first set of segmented electrodes comprising at least two of the segmented electrodes disposed around the circumference of the lead at a first longitudinal position along the lead, and a second set of segmented electrodes comprising at least two of the segmented electrodes disposed around the circumference of the lead at a second longitudinal position along the lead.

4. The method of claim 3, wherein the segmented electrodes of the first and second sets of segmented electrodes are aligned with each other.

5. The method of claim 3, wherein the segmented electrodes of the first and second sets are staggered with respect to each other.

6. The method of claim 1, wherein the plurality of electrodes further comprises at least one ring electrode.

7. The method of claim 6, wherein at least some of the segmented electrodes are formed into a first set of segmented electrodes comprising at least two of the segmented electrodes disposed around a circumference of the lead at a first longitudinal position along the lead, and a second set of segmented electrodes comprising at least two of the segmented electrodes disposed around a circumference of the lead at a second longitudinal position along the lead.

8. The method of claim 6, wherein the at least one ring electrode comprises a first ring electrode located distal to the plurality of segmented electrodes and a second ring electrode located proximal to the plurality of segmented electrodes.

9. The method of claim 1, wherein the plurality of electrodes further comprises a tip electrode.

10. The method of claim 1, wherein implanting the electrical stimulation lead comprises implanting the electrical stimulation lead so that the lead forms an angle of at least 45° with respect to a dorsal root extending from the dorsal root ganglion.

11. The method of claim 1, wherein implanting the electrical stimulation lead comprises implanting the electrical stimulation lead so that the lead forms an angle of no more than 25° with respect to a dorsal root extending from the dorsal root ganglion.

12. The method of claim 1, wherein implanting the electrical stimulation lead comprises implanting the lead around at least a portion of the dorsal root ganglion with the distal end of the lead formed into a hook shape situated around the portion of the dorsal root ganglion.

13. The method of claim 1, wherein implanting the electrical stimulation lead comprises implanting the lead around at least a portion of the dorsal root ganglion with the distal end of the lead formed into a coil shape situated around the portion of the dorsal root ganglion.

14. The method of claim 1, wherein the segmented electrodes are arranged in at least one helix around the lead.

15. The method of claim 1, wherein the segmented electrodes are all disposed on a same side of the lead.

16. The method of claim 1, wherein the lead further comprises at least one marker or indicia configured and arranged to convey to a practitioner an orientation of the segmented electrodes on the lead.

17. The method of claim 1, wherein applying electrical stimulation to the dorsal root ganglion comprises applying electrical stimulation to the dorsal root ganglion using at least two of the plurality of segmented electrodes of the electrical stimulation lead.

18. The method of claim 6, wherein applying electrical stimulation to the dorsal root ganglion comprises applying electrical stimulation to the dorsal root ganglion using at least one of the plurality of segmented electrodes of the electrical stimulation lead and at least one of the at least one ring electrode of the electrical stimulation lead.

19. An electrical stimulation lead, comprising
a lead body having a distal end, a proximal end, a longitudinal length, and a circumference;
a plurality of electrodes disposed along the distal end of the lead body, wherein the plurality of electrodes comprises a plurality of segmented electrodes, each of the segmented electrodes extending around no more than 75% of the circumference of the lead body;
a plurality of terminals disposed along the proximal end of the lead body; and
a plurality of conductors, each conductor electrically coupling at least one of the electrodes to at least one of the terminals;
wherein the electrical stimulation lead is configured and arranged for implantation near, and stimulation of, a dorsal root ganglion.

20. An electrical stimulation system, comprising:
the electrical stimulation lead of claim 19; and
a control module coupleable to the electrical stimulation lead and configured and arranged for providing stimulation current to patient tissue via the electrical stimulation lead.
